(19) United States
(12) Patent Application Publication
(10) Pub. No.: US 2025/0375236 A1
(43) Pub. Date: Dec. 11, 2025

(54) ABLATION CATHETERS WITH INDUCTION HEATING TO TREAT VARICOSE VEINS

(71) Applicant: Boston Scientific Medical Device Limited, Galway (IE)

(72) Inventors: Yinghua Wang, Shanghai (CN); Mengxiang Luo, Shanghai (CN); Mingfeng Frank Xie, Shanghai (CN); Cheng Zhang, Shanghai (CN); Longsheng Cai, Shanghai (CN); Linshanzi Pan, Shanghai (CN)

(21) Appl. No.: 18/881,641

(22) PCT Filed: Jul. 13, 2023
(86) PCT No.: PCT/EP2023/069515

§ 371 Date: Jan. 6, 2025

(30) Foreign Application Priority Data
Jul. 14, 2022 (CN) 202210834218.1

(51) Int. Cl. A61B 18/08; A61B 18/00

(52) U.S. Cl. CPC A61B 18/082; A61B 2018/00077; A61B 2018/00095; A61B 2018/00101; A61B 2018/00404; A61B 2018/00577; A61B 2018/00702

(57) ABSTRACT

At least some embodiments of the present disclosure are directed to a catheter for use in varicose vein treatment including a handle, an elongated shaft connected to the handle, and a heating element disposed near the distal end of the shaft. In some embodiments, the heating element includes a tubular conductor formed from a magnetic material and connected to the elongated shaft, and an inductive coil helically wound over the tubular conductor.

### TECHNICAL FIELD

The present disclosure pertains to medical devices, systems, and methods for providing a therapeutic heat treatment. More particularly, the present disclosure pertains to medical devices, systems and methods for providing therapeutic heat treatments to venous diseases.

### BACKGROUND

Therapeutic heat treatment can be used to treat a wide variety of medical conditions such as tumors, fungal growth, etc. Heat treatments can be used for treating medical conditions alongside other therapeutic approaches or as a standalone therapy. Heat treatment provides localized heating and thus does not cause any cumulative toxicity in contrast to other treatment methods such as drug-based therapy, for example.

One exemplary clinical application of therapeutic heat treatment is in the treatment of chronic venous diseases such as varicose veins, which may become enlarged and/or tortuous due to one or more pathological conditions. Application of sufficient thermal energy via an intravascular device can treat varicose veins by constricting or occluding the target veins.

There is a continuing need for improved devices and methods to provide focused, controlled thermal energy for thermally treating chronic venous conditions such as varicose veins while minimizing or eliminating effects on surrounding healthy tissue.

### SUMMARY

In Example 1, a device for treating varicose veins includes a catheter having an elongated shaft having a proximal end and a distal end, and a heating element disposed near the distal end of the elongated shaft. The elongated shaft may be sized and configured such that the distal end can be inserted into a blood vessel; and the heating element may include a tubular conductor formed from a magnetic material and connected to the elongated shaft, an inductive coil helically wound over the tubular conductor, and a dielectric layer disposed between the tubular conductor and the inductive coil

In Example 2, the device of Example 1, wherein the inductive coil is configured to generate an electromagnetic induction field around the tubular conductor, wherein the tubular conductor is configured to generate thermal energy sufficient for ablation.

In Example 3, the device of either Examples 1 or 2, wherein the dielectric layer includes an insulative coating disposed on the inductive coil.

In Example 4, the device of any of Examples 1-3, wherein the heating element includes a set of tubular conductors, the set of tubular conductors having the tubular conductor and one or more additional tubular conductors, the set of tubular conductors longitudinally spaced from one another along the shaft.

In Example 5, the device of Example 4, wherein the heating element further includes one or more non-conductive tubular sections, at least one non-conductive tubular section disposed between two adjacent tubular conductors of the set of tubular conductors.

In Example 6, the device of Example 5, wherein the one or more non-conductive tubular sections are flexible.

In Example 7, the device of Example 6, wherein at least one non-conductive tubular section of the one or more non-conductive tubular section is configured to allow a bending angle of greater than 30 degree between two adjacent tubular conductors.

In Example 8, the device of any of Examples 1-7, wherein the tubular conductor includes stainless steel or carbon steel.

In Example 9, the device of any of Examples 1-8, wherein the inductive coil includes electrically conductive material.

In Example 10, the device of any of Examples 1-9, wherein the inductive coil includes varnished copper wire.

In Example 11, the device of any of Examples 1-10, wherein the dielectric layer is configured to withstand high temperature and to insulate the tubular conductor and the inductive coil.

In Example 12, the device of any of Examples 1-11, wherein the dielectric layer includes polyimide.

In Example 13, a system for treating varicose veins includes the device of any of Examples 1-12, an energy generator connected to the elongated catheter and configured to generate an electric signal; and a controller operatively connected to the energy generator to control the generation of the electric signal.

In Example 14, the system of Example 13, wherein the inductive coil of the heating element is electrically connected to the energy generator.

In Example 15, the system of Example 14, wherein the heating element includes a set of tubular conductors longitudinally spaced from one another along the shaft; wherein the inductive coil includes a plurality of coil segments individually connected to the energy generator; wherein each coil segment of the plurality of coil segments is individually controllable and addressable.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic illustration of an exemplary ablation device for treating chronic venous diseases, e.g., varicose veins, according to an embodiment of the present disclosure.

FIG. 2A is a schematic illustration of an exemplary ablation catheter including a connector for treating chronic venous diseases, e.g., varicose veins, according to an embodiment of the present disclosure.

FIG. 2B is a schematic cross-sectional view of the connector of the exemplary ablation catheter of FIG. 2A, according to embodiments of the present disclosure.

FIGS. 3A and 3B are schematic elevation and partial cross-sectional views, respectively, of the distal end portion of an ablation catheter, according to embodiments of the present disclosure.

FIGS. 4A-4D are schematic elevation, cross-sectional, partial blown-up, and elevation views, respectively, of the distal end portion of an ablation catheter, according to embodiments of the present disclosure.

FIGS. 5A and 5B are schematic illustrations of a portion of an ablation catheter for use in a target blood vessel in a patient for treatment of varicose veins, according to embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, and/or dimensions are provided for selected elements. Those skilled in the art will recognize that many of the noted examples have a variety of suitable alternatives.

Therapeutic heat treatment can be used to treat a wide variety of medical conditions including chronic venous diseases such as varicose veins, which may become enlarged and/or tortuous due to one or more pathological conditions. Application of sufficient thermal energy via an intravascular device can treat varicose veins by constricting or occluding the target veins.

An exemplary catheter for use in varicose vein treatment may include a handle, an elongated shaft connected to the handle, and a heating element disposed near the distal end of the shaft. In some embodiments, the heating element may receive currents (e.g., alternating currents, direct currents) delivered by an energy generator to generate and deliver thermal ablative energy. In certain embodiments, the heating element may receive electrical signals (e.g., radiofrequency alternating currents) generated by an energy generator to generate and deliver radiofrequency ablative energy.

As mentioned above, there is a continuing need for improved devices and methods to provide focused, controlled thermal energy for thermally treating chronic venous conditions such as varicose veins while minimizing or eliminating effects on surrounding healthy tissue. For example, increased flexibility is desired on the catheter used to treat target blood vessel to minimize potential undesirable harm to vessel walls during treatment. Alternative ways of providing thermal energy for the treatment is also desired for improved and diversified treatment methods. In some instances, a way of increasing the speed of heat generation is desired.

Some embodiments of the present disclosure describe a catheter with an elongated shaft having a proximal end and a distal end and a heating element disposed near the distal end of the shaft. In some embodiments, the heating element may include a tubular conductor formed from a magnetic material and connected to the elongated shaft, an inductive coil helically wound over the tubular conductor, and a dielectric layer disposed between the tubular conductor and the inductive coil. In some embodiments, the heating element may include a plurality of tubular conductors formed from a magnetic material longitudinally spaced from one another along the shaft and connected to the elongated shaft, an inductive coil helically wound over the tubular conductor, and a dielectric layer disposed between the tubular conductor and the inductive coil.

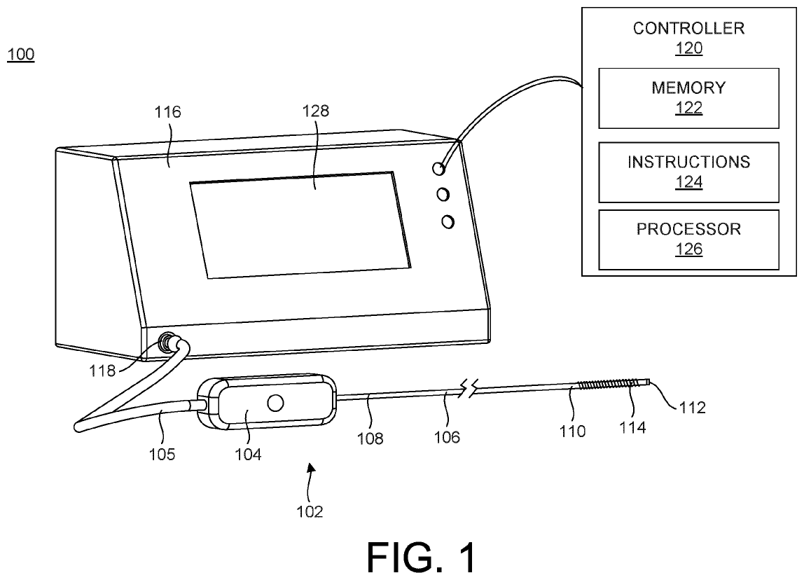

FIG. 1 is a schematic illustration of an exemplary ablation device 100 for treating chronic venous diseases, e.g., varicose veins, according to an embodiment of the present disclosure. The ablation device 100 includes an ablation catheter 102 including a handle 104, an elongated shaft 106 having a proximal end 108 and a distal end portion 110 terminating at a distal end 112, and a heating element 114 disposed near the distal end 112 of the elongated shaft 106. The shaft 106 is sized and configured such that the distal end 112 may be inserted into a target blood vessel. The heating element 114 is configured to deliver ablative energy (e.g., radiofrequency energy, thermal energy) to walls of a target blood vessel.

The ablation device 100 may include an energy generator 116 electrically coupled to the handle 104 via a connector 118 and configured to generate energy by delivering an electric signal (e.g., currents, radiofrequency alternating currents). A controller 120 is operatively connected to the energy generator 116 to control the generation of the electric signal. The controller 120 can be implemented using firmware, integrated circuits, and/or software modules that interact with each other or are combined together. For example, the controller 120 may include memory 122 storing computer-readable instructions/code 124 for execution by a processor 126 (e.g., microprocessor) to perform aspects of embodiments of methods discussed herein.

According to certain embodiments, the heating element 114 employs structural features and/or components to improve the clinical performance as well as enhance the manufacturability of the ablation catheter 102. In some embodiments, the heating element 114 may include a tubular conductor formed from a magnetic material and connected to the elongated shaft 106, an inductive coil helically wound over the tubular conductor, and a dielectric layer disposed between the tubular conductor and the inductive coil. In some embodiments, the heating element 114 may include a plurality of tubular conductors formed from a magnetic material longitudinally spaced from one another along the shaft 106 with at least one of the plurality of tubular conductors extended from the shaft 106, an inductive coil helically wound over the tubular conductor, and a dielectric layer disposed between the tubular conductor and the inductive coil. In certain embodiments, two adjacent tubular conductors have a non-conductive tubular section in between. In some embodiments, the inductive coil includes a plurality of coil segments, where each coil segment is proximate to a corresponding tubular conductor.

In certain embodiments, the heating element 114 may include one or more non-conductive tubular sections, at least one non-conductive tubular section disposed between two adjacent tubular conductors of the plurality of tubular conductors. In some embodiments, the heating element 114 includes a non-conductive tubular section disposed between every two adjacent tubular conductors of the plurality of tubular conductors. In certain embodiments, the dielectric layer is disposed on the tubular conductor to provide electrical insulation. In some embodiments, the dielectric layer includes a material with relatively high thermal conductivity. In certain embodiments, the dielectric layer is disposed on the inductive coil.

In embodiments, the inductive coil may be connected to the energy generator 116 by the handle 104 and cable 105. In some embodiments, the controller 120 may be configured to communicate with various components of the device 100 and generate a graphical user interface (GUI) to be displayed via a display 128.

The controller 120 may include any type of computing device suitable for implementing embodiments of the disclosure. Examples of computing devices include specialized computing devices or general-purpose computing devices such as workstations, servers, laptops, portable devices, desktop, tablet computers, hand-held devices, general-purpose graphics processing units (GPGPUs), and the like, all of which are contemplated within the scope of FIG. 1 with reference to various components of the device 100.

In some embodiments, the controller 120 includes a bus that, directly and/or indirectly, couples the following devices: a processor, a memory, an input/output (I/O) port, an I/O component, and a power supply. Any number of additional components, different components, and/or combinations of components may also be included in the computing device. The bus represents what may be one or more busses (such as, for example, an address bus, data bus, or combination thereof). Similarly, in some embodiments, the computing device may include a number of processors, a number of memory components, a number of I/O ports, a number of I/O components, and/or a number of power supplies. Additionally, any number of these components, or combinations thereof, may be distributed and/or duplicated across a number of computing devices.

In some embodiments, the memory 122 includes computer-readable media in the form of volatile and/or nonvolatile memory, transitory and/or non-transitory storage media and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In some embodiments, the memory 122 stores computer-executable instructions for causing a processor (e.g., the controllers 120) to implement aspects of embodiments of system components discussed herein and/or to perform aspects of embodiments of methods and procedures discussed herein.

The computer-executable instruction 124 may include, for example, computer code, machine-useable instructions, and the like such as, for example, program components capable of being executed by one or more processors associated with a computing device. Program components may be programmed using any number of different programming environments, including various languages, development kits, frameworks, and/or the like. Some or all of the functionality contemplated herein may also, or alternatively, be implemented in hardware and/or firmware.

In some embodiments, the memory 122 may include a data repository implemented using any one of the configurations described below. A data repository may include random access memories, flat files, XML files, and/or one or more database management systems (DBMS) executing on one or more database servers or a data center. A database management system may be a relational (RDBMS), hierarchical (HDBMS), multidimensional (MDBMS), object oriented (ODBMS or OODBMS) or object relational (ORDBMS) database management system, and the like. The data repository may be, for example, a single relational database. In some cases, the data repository may include a plurality of databases that can exchange and aggregate data by data integration process or software application. In an exemplary embodiment, at least part of the data repository may be hosted in a cloud data center. In some cases, a data repository may be hosted on a single computer, a server, a storage device, a cloud server, or the like. In some other cases, a data repository may be hosted on a series of networked computers, servers, or devices. In some cases, a data repository may be hosted on tiers of data storage devices including local, regional, and central.

Various components of the device 100 can communicate via or be coupled to via a communication interface, for example, a wired or wireless interface. The communication interface includes, but not limited to, any wired or wireless short-range and long-range communication interfaces. The wired interface can use cables, umbilicals, and the like. The short-range communication interfaces may be, for example, local area network (LAN), interfaces conforming known communications standard, such as Bluetooth® standard, IEEE 702 standards (e.g., IEEE 702.11), a ZigBee® or similar specification, such as those based on the IEEE 702.15.4 standard, or other public or proprietary wireless protocol. The long-range communication interfaces may be, for example, wide area network (WAN), cellular network interfaces, satellite communication interfaces, etc. The communication interface may be either within a private computer network, such as intranet, or on a public computer network, such as the internet.

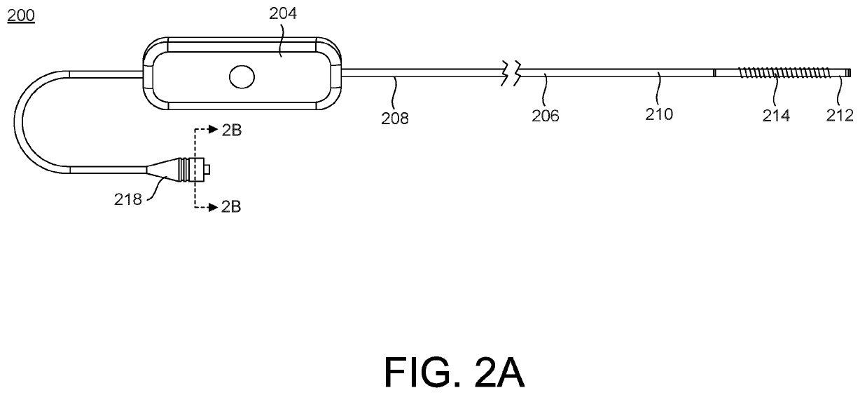

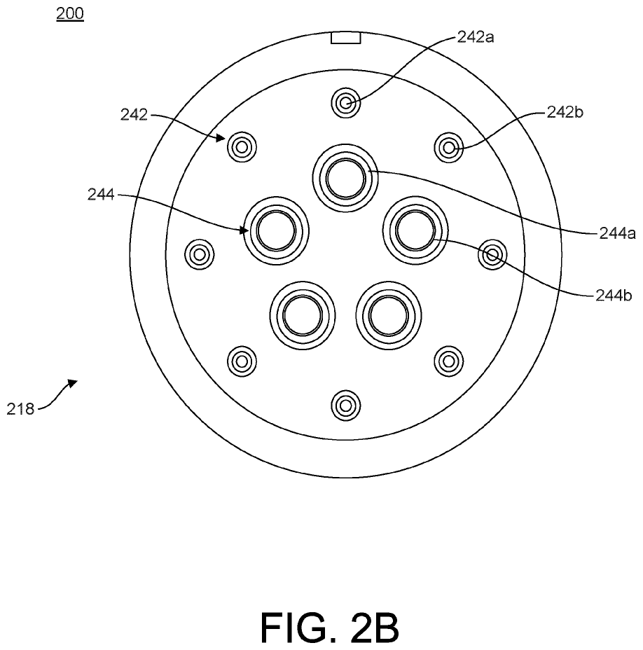

FIG. 2A is a schematic illustration of an exemplary ablation catheter 200 including a connector 218 (similar to the connector 118 as shown in FIG. 1) for treating chronic venous diseases, e.g., varicose veins; FIG. 2B is a schematic cross-sectional view of the connector 218 of the exemplary ablation catheter 200 along the cross-sectional indicator lines 2B-2B of FIG. 2A, according to embodiments of the present disclosure.

As shown, the ablation catheter 200 includes a handle 204, an elongated shaft 206 having a proximal end 208 and a distal end portion 210 terminating at a distal end 212, and a heating element 214 disposed near the distal end 212 of the elongated shaft 206. The shaft 206 is sized and configured such that the distal end 212 may be inserted into a target blood vessel. The heating element 214 is configured to deliver ablative energy (e.g., radiofrequency energy, thermal energy) to the wall of a target blood vessel.

In some embodiments, the connector 218 includes pins of different sizes 242 (including e.g., pins 242a, 242b) and 244 (including e.g., pins 244a, 244b). The pins 242 are relatively smaller than pins 244, and are configured to transfer electric signals (e.g., the electric signal generated by the energy generator 116 in FIG. 1). Exemplary electric signals may include thermocouple signals or pressure signals. The pins 244 are relatively larger compared to pins 242, and may be configured to allow current to pass from an energy generator (e.g., the energy generator 116 in FIG. 1) to generate heat on the heating element 214. One of the pins 244 may be used as a pin connected to ground (i.e., a ground pin). In some embodiments, where the heating elements include multiple heating segments (e.g., coil segments), the ground pin may be used as a common ground pin by the multiple heating segments.

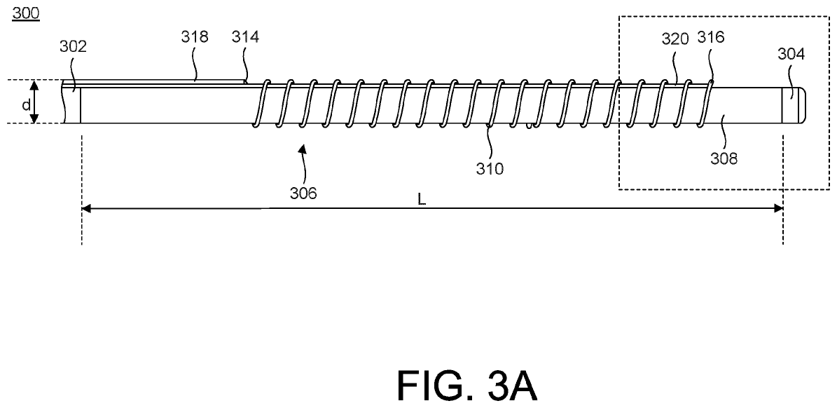

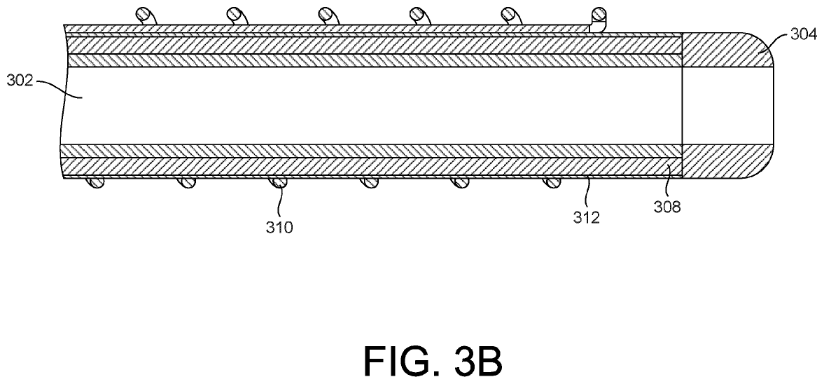

FIGS. 3A and 3B are schematic elevation and partial cross-sectional views, respectively, of the distal end portion of an ablation catheter, according to embodiments of the present disclosure. As shown, the distal end portion 300 includes a part of an elongate shaft 302 terminating at a distal end 304, and a heating element 306 disposed near the distal end 304 of the elongated shaft 302. The shaft 302 is sized and configured such that the distal end 304 may be inserted into a target blood vessel.

The heating element 306 includes a tubular conductor 308 formed from a magnetic material and connected to the elongated shaft 302, an inductive coil 310 helically wound over the tubular conductor 308, and a dielectric layer 312 disposed between the tubular conductor 308 and the inductive coil 310.

In some embodiments, the inductive coil 310 is operatively connected to an energy generator (e.g., the energy generator 116 in FIG. 1) and configured to generate thermal energy on the tubular conductor by electromagnetic induction. As will be understood by a skilled artisan, induction heating is the process of heating an electrically conducting object (e.g., the tubular conductor 308) by electromagnetic induction, through heat generated in the electrically conducting object by eddy currents. Induction heating occurs when an electromagnetic force field produces an electrical current in a metal part (e.g., the tubular conductor 308), and the surface of the metal part heats due to the resistance to the flow of the electric current. In embodiments, the induction generator or heater (e.g., the inductive coil 310) is shaped to contour the metal part (e.g., the tubular conductor 308).

In some embodiments, the inductive coil 310 is electrically insulated from the tubular conductor 308 with an insulative coating disposed on the inductive coil. In some embodiments, the tubular conductor 308 may be made of magnetically conductive material (e.g., stainless steel or carbon steel). In some embodiments, the inductive coil 310 may be made of electrically conductive material (e.g., varnished copper wire). In some embodiments, the dielectric layer 312 is configured to withstand high temperature and to insulate the tubular conductor 308 and the inductive coil 310. In an exemplary embodiment, the dielectric layer 312 may include polyimide.

In an exemplary embodiment, for example as shown in FIG. 3A, a first end 314 and a second end 316 of the inductive coil 310 may be combined together and connected to an induction heater output interface with wires 318 and 320. In some embodiments, the inductive coil 310 may be operatively connected to an energy generator (e.g., the energy generator 116 in FIG. 1) through a handle (e.g., the handle 104 in FIG. 1) and a cable (e.g. the cable 105 in FIG. 1). In embodiments, the tubular conductor 308 is sized to be inserted into a target vessel while providing ablation efficiency (e.g., sufficiently wide, sufficiently long, etc.). In some embodiments, the length (L) of the tubular conductor 308 may be from about three (3) centimeters to about seven (7) centimeters long. In some embodiments, the diameter (d) of the inductive coil 310 surrounding the tubular conductor 308 may be from about one and a half (1.5) millimeters to about eighteen (18) millimeters. In certain embodiments, the length of the tubular conductor 308 is greater than two (2) centimeters. In some embodiments, the length of the tubular conductor is less than ten (10) centimeters. In certain embodiments, the diameter of the tubular conductor 308 is greater than one (1) millimeter. In some embodiments, the diameter of the tubular conductor 308 is less than twenty (20) millimeters.

One or more pressure sensors (not shown) may be disposed proximate to the heating element 306 to measure signals indicative of pressures applied to the heating element 306 via target tissue (e.g., a target vessel wall). In some embodiments, a plurality of pressure sensors (e.g., three sensors, four sensors, six sensors) are disposed circumferentially about the heating element (e.g., two adjacent pressure sensors are offset by certain degrees from one another in a projected view).

In some embodiments, the plurality of pressure sensors include at least one selected from a group consisting of a piezoelectric pressure sensor, a capacitive pressure sensor, an inductive pressure sensor, a strain gauge pressure sensor, and a potentiometric pressure sensor. According to certain embodiments, during treatment, the heating element 306 is controlled to deliver ablative energy when an output signal indicative of pressure generated by at least one pressure sensor of the plurality of pressure sensors is greater than a predetermined threshold. In certain embodiments, the heating element 306 is controlled to deliver ablative energy when output signals indicative of pressure generated by a part of all pressure sensors of the plurality of pressure sensors are greater than a predetermined threshold.

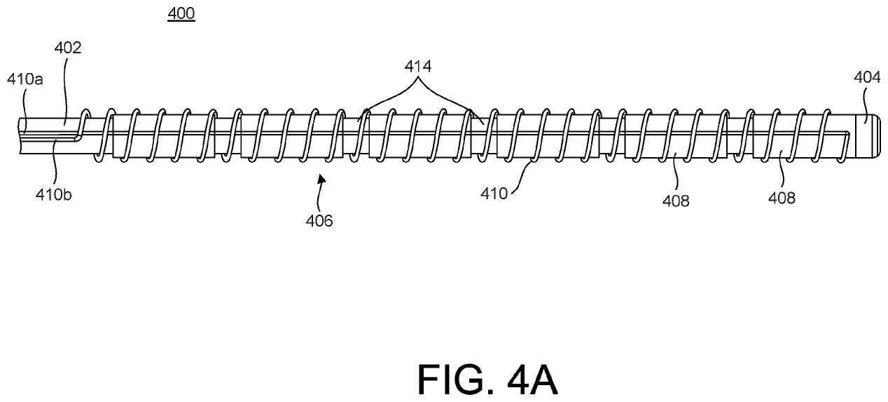

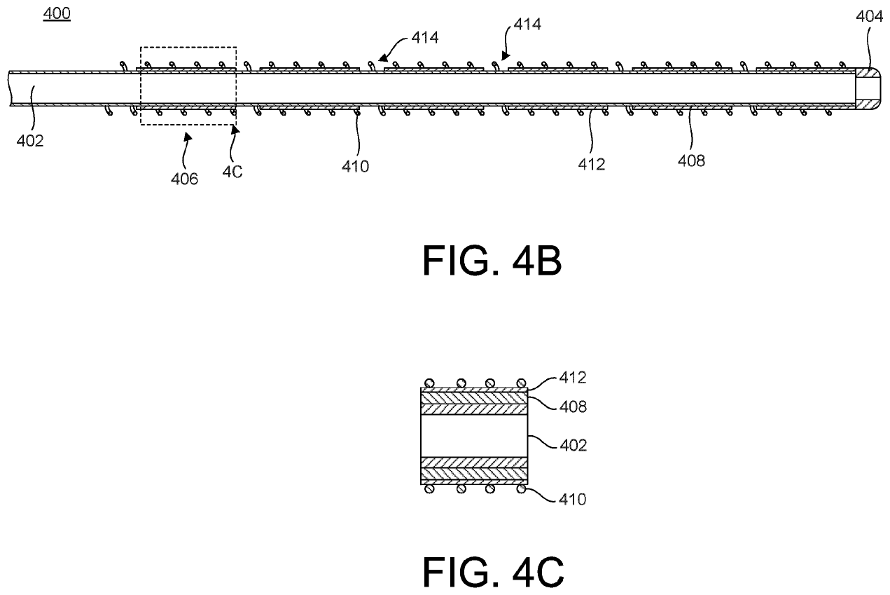

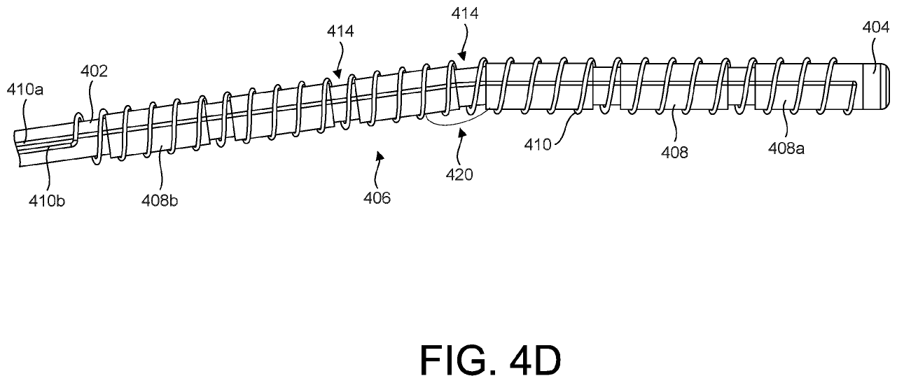

FIGS. 4A-4D are schematic elevation, cross-sectional, partial blown-up, and elevation views, respectively, of a distal end portion of an ablation catheter, according to embodiments of the present disclosure. As shown, the distal end portion 400 includes part of an elongate shaft 402 terminating at a distal end 404, and a heating element 406 disposed near the distal end 404 of the elongated shaft 402. The shaft 402 and/or the heating element 406 is sized and configured such that the distal end 404 may be inserted into a target blood vessel.

The heating element 406 may include a plurality of tubular conductors 408 formed from a magnetic material and connected to the elongated shaft 402. The plurality of tubular conductors 408 are longitudinally spaced from one another along the shaft 402. The heating element 406 may further include an inductive coil 410 helically wound over the tubular conductor 408, and a dielectric layer 412 disposed between the tubular conductor 408 and the inductive coil 410.

In embodiments, the two ends of the inductive coil 410a and 410b are connected to the induction heater output interface respectively. In some embodiments, the inductive coils 410a-b are operatively connected to the energy generator (e.g., the energy generator 116 in FIG. 1) and configured to generate thermal energy on the plurality of tubular conductors 408 by electromagnetic induction. In some embodiments, the inductive coil 410 is electrically insulated from the plurality of tubular conductors 408 with an insulative coating disposed on the inductive coil.

In some embodiments, the plurality of tubular conductors 408 may be made of magnetically conductive material (e.g., stainless steel or carbon steel). In some embodiments, the inductive coil 410 may be made of electrically conductive material (e.g., varnished copper wire). In some embodiments, the dielectric layer 412 includes a dielectric material withstanding high temperature and to insulate the tubular conductor 408 and the inductive coil 410. In an exemplary embodiment, the dielectric layer 412 may include polyimide. In certain embodiments, the dielectric layer 412 includes a dielectric material having relatively high thermal conductivity.

As shown, the plurality of tubular conductors 408 are longitudinally spaced from one another along the shaft 402. In embodiments, the heating element 406 may include one or more non-conductive tubular sections 414, at least one non-conductive tubular section 414 disposed between two adjacent tubular conductors of the set of tubular conductors 408. In some embodiments, the one or more non-conductive tubular sections 414 are flexible such that at least one non-conductive tubular section of the one or more non-conductive tubular sections 414 is configured to allow a bending angle 420 of greater than 30 degree between two adjacent tubular conductors. During treatment, for example as shown in FIG. 4C, the elongated shaft 402 may bend at one of the one or more gaps or non-conductive tubular sections 414 while being inserted into the target vessel to better fit the contour of the vessel. In embodiments, the elongated shaft 402 may bend at a plurality of the gaps or one or more non-conductive tubular sections 414.

As veins may become tortuous due to chronic venous diseases, it is not easy for operators to insert the distal end portion 400 of an ablation catheter into the target vein. Placement of the heating element 406 on the distal end portion 400 to a specific treatment site may become increasingly difficult if the catheter is too stiff. Increasing flexibility of the catheter makes it easier for the distal end portion 400 to go through tortuous veins and arrive at target treatment site, and may also reduce the operation time. In addition, using inductive coil 410 and tubular conductors 408 instead of a heat resistant coil may increase the speed of heat generation as induction heating typically has fast response to energy generator input.

In embodiments, the heating element 406 and the tubular conductors 408 are sized to be inserted into a target vessel while providing ablation efficiency (e.g., sufficiently wide, sufficiently long, etc.). In some embodiments, the length (L) of the plurality of tubular conductors collectively 408 may be from about three (3) centimeters to about seven (7) centimeters long. In some embodiments, the diameter (d) of the inductive coil 410 surrounding the tubular conductors 408 may be from about one and a half (1.5) millimeters to about eighteen (18) millimeters. In certain embodiments, the length of the plurality of tubular conductors is greater than two (2) centimeters. In some embodiments, the length of the plurality of tubular conductors is less than ten (10) centimeters. In certain embodiments, the diameter of the tubular conductor 408 is greater than one (1) millimeter. In some embodiments, the diameter of the tubular conductor 408 is less than twenty (20) millimeters.

In certain embodiments, the inductive coil 410 is an integrated section, and the plurality of tubular conductors may be heated simultaneously during treatment. In some embodiments (not shown), the inductive coil 410 includes a plurality of coil segments individually connected to an energy generator, and each coil segment is individually controllable and addressable. In certain embodiments, at least one tubular conductor 408a is heated and at least one tubular conductor 408b is not heated during a treatment.

A temperature sensor (not shown) may be disposed in one or more of the gaps or non-conductive tubular sections 414. Based on signals indicative of temperatures measured by the temperature sensor, a controller (e.g., the energy controller 120 in FIG. 1) or a physician may selectively adjust the power delivery to the inductive coil 410, thus adjusting heat delivered to the target blood vessel.

One or more pressure sensors (not shown) may be disposed proximate to the heating element 406 and/or the plurality of tubular conductors 408 to measure signals indicative of pressures applied to the heating element 406 via target tissue (e.g., a target vessel wall). In some embodiments, a plurality of pressure sensors (e.g., three sensors, four sensors, six sensors) are disposed circumferentially about the heating element (e.g., two adjacent pressure sensors are offset by certain degrees from one another in a projected view).

In some embodiments, the plurality of pressure sensors include at least one selected from a group consisting of a piezoelectric pressure sensor, a capacitive pressure sensor, an inductive pressure sensor, a strain gauge pressure sensor, and a potentiometric pressure sensor. According to certain embodiments, during treatment, the heating element 406 and/or the inductive coil 410 is controlled to deliver ablative energy when an output signal indicative of pressure generated by at least one pressure sensor of the plurality of pressure sensors is greater than a predetermined threshold. In certain embodiments, the heating element 406 and/or the inductive coil 410 is controlled to deliver ablative energy when output signals indicative of pressure generated by a part of all pressure sensors of the plurality of pressure sensors are greater than a predetermined threshold.

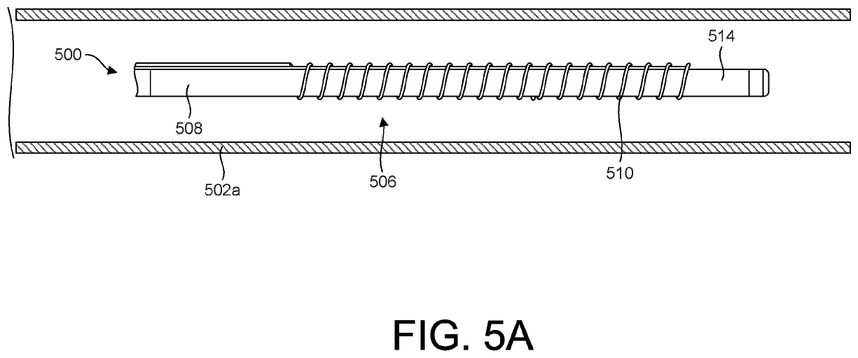

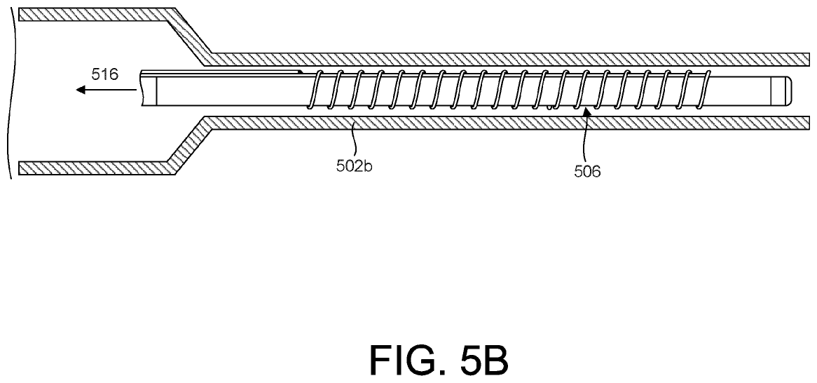

FIGS. 5A and 5B are schematic illustrations of a portion of an ablation catheter for use in a target blood vessel in a patient for treatment of varicose veins, according to embodiments of the present disclosure.

In some embodiments, during endovenous thermal ablation procedure, an introducer sheath may be positioned inside a patient's target vein using ultrasonic guidance and standard vascular technique. An ablation catheter (e.g., the ablation catheter 102 in FIG. 1) may then be inserted into the target vein through the introducer sheath. In some circumstances, under ultrasonic guidance, tumescent anesthetic solution or saline may be injected into target vein segment to act as a heat sink that protects tissue from thermal injury, and improve thermal conductivity between the wall of target vein and the ablation catheter.

As shown in FIG. 5A, the distal end portion 500 of an ablation catheter (e.g., the ablation catheter 102 in FIG. 1) is positioned in a target blood vessel 502a. The ablation catheter may be introduced and positioned with an introducer sheath using ultrasonic guidance. As will be appreciated by a skilled artisan, any standard vascular technique may be used here to introduce and position the distal end portion 500 of the ablation catheter into the target vein segment. The distal end portion 500 may include a heating element 506 having an inductive coil 510 helically wound over a tubular conductor 508, and a dielectric layer 514 disposed between the tubular conductor 508 and the inductive coil 510.

In some embodiments, during treatment, current may be applied to the inductive coil 510 by a generator (e.g., the energy generator 116 in FIG. 1), and a segment of the target blood vessel 502a adjacent the heating element 506. The generator may include a radiofrequency generator that generates radiofrequency current to heat the tubular conductor 508 and the target blood vessel 502a adjacent the tubular conductor 508. During treatment, the tubular conductor 508 is heated up by electromagnetic induction, and the target blood vessel may start to close or reduce in diameter, shown as 502b in FIG. 5B.

In some implementations, the ablation catheter may include a temperature sensor disposed along the length of a shaft of the catheter, and power delivery to the inductive coil 510 may be adjusted automatically by a controller (e.g., the controller 120 in FIG. 1) based on temperature or signals indicative of temperature measured by the temperature sensor. In some embodiments, the power delivery to the inductive coil 510 may heat the inductive coil 510 to about 80° C. to about 140° C. for treating varicose veins. In some embodiments, the power delivery to the inductive coil 510 may heat the inductive coil 510 to about 100° C. to about 130° C. for treating varicose veins. In some embodiments, the power delivery to the inductive coil 510 may heat the inductive coil 510 to about 120° C. for treating varicose veins.

A segment of the target blood vessel 502a adjacent the tubular conductor 508 being treated will close (e.g., shrink, reduced in diameter) as the conductor is heated up, shown as 502b in FIG. 11B. External pressure may be applied as needed during treatment. After a certain section is treated (i.e. the section of the vein is closed), the catheter may be moved towards the venous access, as indicated by arrow 516, and the process repeated until the entire vein is closed. The catheter and introducer sheath may then be removed after treatment is done. In some use cases, a diameter of the heating element 506 is smaller than a diameter of blood vessel 502a and the heating element 506 can be moved close to the vessel wall during the treatment.

In Example 16, a device for treating varicose veins includes an energy generator and a catheter connected to the energy generator. In some embodiments, the elongated catheter may include an elongated shaft having a proximal end and a distal end, the shaft being sized and configured such that the distal end can be inserted into a blood vessel; and a heating element disposed near the distal end of the elongated shaft. In some embodiments, the heating element may include a plurality of tubular conductors formed from a magnetic material longitudinally spaced from one another along the shaft and connected to the elongated shaft; an inductive coil helically wound over the plurality of tubular conductors; a dielectric layer disposed between the plurality of tubular conductors and the inductive coil; and one or more non-conductive tubular sections, at least one non-conductive tubular section disposed between two adjacent tubular conductors of the set of tubular conductors.

In Example 17, the device of Example 16, wherein the inductive coil is electrically connected to the energy generator and configured to generate an electromagnetic induction field around the plurality of tubular conductors, wherein the plurality of tubular conductors are configured to generate thermal energy sufficient for ablation.

In Example 18, the device of Example 16, wherein the dielectric layer comprises an insulative coating disposed on the inductive coil.

In Example 19, the device of Example 16, wherein the one or more non-conductive tubular sections are flexible.

In Example 20, the device of Example 19, wherein at least one non-conductive tubular section of the one or more non-conductive tubular section is configured to allow a bending angle of greater than 30 degree between two adjacent tubular conductors.

In Example 21, the device of Example 16, wherein the plurality of tubular conductors include stainless steel or carbon steel.

In Example 22, the device of Example 16, wherein the inductive coil includes electrically conductive material.

In Example 23, the device of Example 22, wherein the inductive coil includes varnished copper wire.

In Example 24, the device of Example 16, wherein the dielectric layer is configured to withstand high temperature and to insulate the tubular conductor and the inductive coil.

In Example 25, the device of Example 16, wherein the dielectric layer includes polyimide.

As the terms are used herein with respect to measurements (e.g., dimensions, characteristics, attributes, components, etc.), and ranges thereof, of tangible things (e.g., products, inventory, etc.) and/or intangible things (e.g., data, electronic representations of currency, accounts, information, portions of things (e.g., percentages, fractions), calculations, data models, dynamic system models, algorithms, parameters, etc.), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error; differences in measurement and/or manufacturing equipment calibration; human error in reading and/or setting measurements; adjustments made to optimize performance and/or structural parameters in view of other measurements (e.g., measurements associated with other things); particular implementation scenarios; imprecise adjustment and/or manipulation of things, settings, and/or measurements by a person, a computing device, and/or a machine; system tolerances; control loops; machine-learning; foreseeable variations (e.g., statistically insignificant variations, chaotic variations, system and/or model instabilities, etc.); preferences; and/or the like.

Although illustrative methods may be represented by one or more drawings (e.g., flow diagrams, communication flows, etc.), the drawings should not be interpreted as implying any requirement of, or particular order among or between, various steps disclosed herein. However, certain some embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

1. A device for treating varicose veins, comprising:
a catheter comprising:
an elongated shaft having a proximal end and a distal end, the shaft being sized and configured such that the distal end can be inserted into a blood vessel; and
a heating element disposed near the distal end of the elongated shaft, the heating element comprising a tubular conductor formed from a magnetic material and connected to the elongated shaft, an inductive coil helically wound over the tubular conductor, and a dielectric layer disposed between the tubular conductor and the inductive coil.

2. The device of claim 1, wherein the inductive coil is configured to generate an electromagnetic induction field around the tubular conductor, wherein the tubular conductor is configured to generate thermal energy sufficient for ablation.

3. The device of claim 1, wherein the dielectric layer comprises an insulative coating disposed on the inductive coil.

4. The device of claim 3, wherein the heating element comprises a set of tubular conductors, the set of tubular conductors comprising the tubular conductor and one or more additional tubular conductors, the set of tubular conductors longitudinally spaced from one another along the shaft.

5. The device of claim 4, wherein the heating element further comprises one or more non-conductive tubular sections, at least one non-conductive tubular section disposed between two adjacent tubular conductors of the set of tubular conductors.

6. The device of claim 5, wherein the one or more non-conductive tubular sections are flexible.

7. The device of claim 6, wherein at least one non-conductive tubular section of the one or more non-conductive tubular section is configured to allow a bending angle of greater than 30 degree between two adjacent tubular conductors.

8. The device of claim 7, wherein the tubular conductor includes stainless steel or carbon steel.

9. The device of claim 8, wherein the inductive coil includes electrically conductive material.

10. The device of claim 9, wherein the inductive coil includes varnished copper wire.

11. The device of claim 10, wherein the dielectric layer is configured to withstand high temperature and to insulate the tubular conductor and the inductive coil.

12. The device of claim 11, wherein the dielectric layer includes polyimide.

13. A system for treating varicose veins, comprising:
a catheter comprising:
an elongated shaft having a proximal end and a distal end, the shaft being sized and configured such that the distal end can be inserted into a blood vessel; and
a heating element disposed near the distal end of the elongated shaft, the heating element comprising a tubular conductor formed from a magnetic material and connected to the elongated shaft, an inductive coil helically wound over the tubular conductor, and a dielectric layer disposed between the tubular conductor and the inductive coil;
an energy generator connected to the elongated catheter and configured to generate an electric signal; and
a controller operatively connected to the energy generator to control the generation of the electric signal.

14. The system of claim 13, wherein the inductive coil of the heating element is electrically connected to the energy generator.

15. The system of claim 14, wherein the heating element comprises a set of tubular conductors longitudinally spaced from one another along the shaft; wherein the inductive coil comprises a plurality of coil segments individually connected to the energy generator; wherein each coil segment of the plurality of coil segments is individually controllable and addressable.

16. A catheter for ablation within a varicose vein of a patient, the catheter comprising:
an elongated shaft having a proximal end and a distal end;
a tubular conductor formed from a magnetic material coupled to the distal end of the elongated shaft;
an inductive coil helically wound over the tubular conductor; and
a dielectric layer disposed between the tubular conductor and the inductive coil.

17. The catheter of claim 16, wherein the inductive coil is configured to generate an electromagnetic induction field around the tubular conductor, wherein the tubular conductor is configured to generate thermal energy sufficient for ablation.

18. The catheter of claim 17, wherein the dielectric layer comprises an insulative coating disposed on the inductive coil.

19. The catheter of claim 17, further comprising a set of tubular conductors, the set of tubular conductors including the tubular conductor and one or more additional tubular conductors, the set of tubular conductors longitudinally spaced from one another along the shaft.

20. The catheter of claim 19, further comprising one or more non-conductive tubular sections, at least one non-conductive tubular section disposed between two adjacent tubular conductors of the set of tubular conductors.
